(19) United States
(12) Patent Application Publication
(10) Pub. No.: US 2013/0324632 A1
(43) Pub. Date: Dec. 5, 2013

(54) POLYESTEROLS FOR PRODUCING RIGID POLYURETHANE FOAMS

(71) Applicant: BASF SE, Ludwigshafen (DE)

(72) Inventors: Olaf JACOBMEIER, Luebbecke (DE); Gunnar Kampf, Stemwede-Haldem (DE)

(21) Appl. No.: 13/897,695

(22) Filed: May 20, 2013

Related U.S. Application Data
(60) Provisional application No. 61/652,872, filed on May 30, 2012.

(51) Int. Cl. C08G 18/08; C07C 69/82

(52) U.S. Cl. CPC C08G 18/14; C07C 69/82
USPC 521/172; 554/116; 252/182.27

(57) ABSTRACT

The invention relates to polyesterols obtainable by reaction of
b1) from 10 to 70 mol % of at least one compound selected from the group consisting of terephthalic acid, dimethyl terephthalate, polyethylene terephthalate, phthalic anhydride, phthalic acid and isophthalic acid,
b2) from 0.8 to 4.5 mol % of a fatty acid triglyceride,
b3) from 10 to 70 mol % of a diol selected from the group consisting of ethylene glycol, diethylene glycol and polyethylene glycols,
b4) from 5 to 50 mol % of a polyether polyol having a functionality above 2,
   wherein at least 200 mmol of component b4) are used per kg of the polyesterol, wherein the sum total of components b1) to b4) is 100 mol %.

The invention relates to polyesterols, to a process for producing rigid polyurethane foams using the polyesterols, to the rigid polyurethane foams themselves and also to their use for producing sandwich elements having rigid or flexible outer layers.

The production of rigid polyurethane foams by reacting organic or modified organic di- or polyisocyanates with comparatively high molecular weight compounds having two or more than two reactive hydrogen atoms, especially with polyether polyols from alkylene oxide polymerization or polyester polyols from the polycondensation of alcohols with dicarboxylic acids in the presence of polyurethane catalysts, chain-extending and/or crosslinking agents, blowing agents and further auxiliary and admixture agents is known and has been described in numerous patent and literature publications.

In what follows, the terms “polyester polyol”, “polyesterol”, “polyester alcohol” and the abbreviation “PESOL” are used interchangeably.

Customary polyester polyols are polycondensates of aromatic and/or aliphatic dicarboxylic acids and alkanediols and/or -triols, or ether diols. It is also possible to process polyester scrap, especially polyethylene terephthalate (PET) and/or polybutylene terephthalate (PBT) scrap, into polyester polyols. A whole series of processes are known and have been described for this purpose. Some processes are based on converting the polyester into a diester of terephthalic acid, for example dimethyl terephthalate. DE-A 100 37 14 and U.S. Pat. No. 5,051,528 describe such transesterifications using methanol and transesterification catalysts.

It is also known that esters based on terephthalic acid are superior to esters based on phthalic acid in terms of burning behavior, as described in WO 2010/043624 for example.

When polyester polyols based on aromatic carboxylic acids or derivatives thereof (such as terephthalic acid or phthalic anhydride) are used to produce rigid polyurethane (PU) foams, the high viscosity of the polyester polyols often has a noticeably adverse effect, since the viscosity of blends with the polyesters rises as a result, which makes mixing with the isocyanate distinctly more difficult.

EP-A 1 058 701 discloses aromatic polyester polyols of low viscosity, which are obtained by transesterifying a mixture of phthalic acid derivatives, diols, polyols and hydrophobic fat-based materials.

In addition, certain systems for producing rigid PU foams, for example those employing glycerol as comparatively high-functional alcoholic polyester component, can give rise to problems due to insufficient dimensional stability in that the foamed product distorts significantly after demolding or after the pressure section when processed by the double belt process.

Nor has the problem with the behavior of rigid PU foams in the event of fire hitherto been satisfactorily solved for all systems. For example, a toxic compound can form in the event of fire when using trimethylolpropane (TMP) as comparatively high-functionality alcoholic polyester component.

A general problem with the production of rigid foams is the formation of surface defects, preferentially at the interface with metallic outer layers. These foam surface defects cause formation of an uneven metal surface in sandwich elements and thus often lead to visual unacceptability of the product. An improvement in the foam surface reduces the frequency with which such surface defects occur and thus leads to a visual improvement in the surface of sandwich elements.

Rigid polyurethane foams frequently display high brittleness on cutting with severe evolution of dust and high sensitivity on the part of the foam, and also on sawing where particularly the sawing of composite elements with metallic outer layers and a core of polyisocyanurate foam can lead to crack formation in the foam.

It is further generally desirable to provide systems having a very high self-reactivity in order that the use of catalysts may be minimized.

Smoke gas evolution by rigid polyurethane or polyisocyanurate foam insulants in the event of a fire is problematical.

The invention thus has for its object to provide polyesterols for the production of rigid polyurethane or polyisocyanurate foams which in the event of a fire result in reduced smoke gas evolution by the rigid polyurethane or polyisocyanurate foams produced therewith. The invention further has for its object to provide rigid polyurethane or polyisocyanurate foams having reduced smoke gas evolution in the event of a fire.

This object is achieved by a polyesterol B) obtainable by reaction of
b1) from 10 to 70 mol %, preferably from 20 to 60 mol %, more preferably from 25 to 50 mol % and especially from 30 to 40 mol % of at least one compound selected from the group consisting of terephthalic acid, dimethyl terephthalate, polyethylene terephthalate, phthalic anhydride, phthalic acid and isophthalic acid,
b2) from 0.8 to 4.5 mol %, preferably from 1.0 to 3.8 mol %, more preferably from 1.1 to 3.2 mol % and especially from 1.2 to 2.5 mol % and specifically from 1.3 to 2.0 mol % of a fatty acid triglyceride,
b3) from 10 to 70 mol %, preferably from 20 to 60 mol %, more preferably from 30 to 55 mol % and especially from 40 to 50 mol % of a diol selected from the group consisting of ethylene glycol, diethylene glycol and polyethylene glycol,
b4) from 5 to 50 mol % preferably from 10 to 40 mol %, more preferably from 12 to 30 mol % and especially from 14 to 25 mol % of a polyether polyol having a functionality above 2, wherein at least 200 mmol of component b4) are used per kg of polyesterol B),
   wherein the sum total of components b1) to b4) is 100 mol %.

The object is further achieved by a process for producing rigid polyurethane foams comprising the reaction of
A) at least one polyisocyanate,
B) at least one polyesterol obtainable by reaction of
   b1) from 10 to 70 mol %, preferably from 20 to 60 mol %, more preferably from 25 to 50 mol % and especially from 30 to 40 mol % of at least one compound selected from the group consisting of terephthalic acid, dimethyl terephthalate, polyethylene terephthalate, phthalic anhydride, phthalic acid and isophthalic acid,
   b2) from 0.8 to 4.5 mol %, preferably from 1.0 to 3.8 mol %, more preferably from 1.1 to 3.2 mol % and especially from 1.2 to 2.5 mol % and specifically from 1.3 to 2.0 mol % of a fatty acid triglyceride,
   b3) from 10 to 70 mol %, preferably from 20 to 60 mol %, more preferably from 30 to 55 mol % and especially from 40 to 50 mol % of a diol selected from the group consisting of ethylene glycol, diethylene glycol and polyethylene glycol,
   b4) from 5 to 50 mol % preferably from 10 to 40 mol %, more preferably from 12 to 30 mol % and especially from 14 to 25 mol % of a polyether polyol having a functionality above 2,
   wherein at least 200 mmol of component b4) are used per kg of polyesterol B),
   wherein the sum total of components b1) to b4) is 100 mol %,
C) optionally one or more further polyester polyols other than those of component B),
D) optionally one or more polyether polyols,
E) optionally one or more flame retardants,
F) one or more blowing agents,
G) one or more catalysts, and
H) optionally further auxiliaries or admixture agents.

The present invention also provides a polyol component comprising the aforementioned components B) to H), wherein the mass ratio of total components B) and optionally C) to component D) is at least 1.

The present invention further provides rigid polyurethane foams obtainable by the process of the present invention and also their use for producing sandwich elements having rigid or flexible outer layers. For the purposes of the present invention, rigid polyurethane foams are also to be understood as meaning rigid polyisocyanurate foams, which are specific rigid polyurethane foams.

The embodiments recited hereinbelow in the context of components B) to H) relate not only to the process of the present invention and the rigid foams thus obtainable but also to the polyol component of the present invention.

### Component B

Hereinbelow the terms “polyester polyol” and “polyesterol” are used interchangeably as are the terms “polyether polyol” and “polyetherol”.

Component b1) preferably comprises at least one compound from the group consisting of terephthalic acid (TPA), dimethyl terephthalate (DMT), polyethylene terephthalate (PET), phthalic anhydride (PA) and phthalic acid, more preferably consisting of terephthalic acid (TPA), dimethyl terephthalate (DMT) and polyethylene terephthalate (PET). It is particularly preferable for component b1) to comprise at least one compound from the group consisting of terephthalic acid and dimethyl terephthalate (DMT). Component b1) consists specifically of terephthalic acid. Terephthalic acid and/or DMT in component b1) lead to polyesters B) having particularly good fire protection properties.

The amount in which component b2) is used is preferably from 1.0 to 3.8 mol %, more preferably from 1.1 to 3.2 mol %, more specifically from 1.2 to 2.5 mol %, and even more specifically from 1.3 to 2.0 mol %. The fatty acid triglyceride is preferably soybean oil, rapeseed oil, tallow or a mixture thereof. In a specific embodiment, soybean oil is concerned. In a further specific embodiment, beef tallow is concerned. The fatty acid triglyceride serves inter alia to improve the blowing agent solubility in the production of rigid polyurethane foams, although a comparatively low amount of fatty acid triglyceride b2) in polyesterol B) surprisingly has a favorable effect on smoke gas density in the event of a fire, i.e., smoke gas density decreases.

The amount in which diol b3) is used is preferably from 20 to 60 mol %, more preferably from 30 to 55 mol % and especially from 40 to 50 mol %. This diol is preferably at least one compound from the group consisting of polyethylene glycol (PEG), diethylene glycol (DEG) and monoethylene glycol (MEG), particular preference being given to diethylene glycol (DEG) and monoethylene glycol (MEG) and especial preference to diethylene glycol (DEG). Polyethylene glycol is to be understood as meaning triethylene glycol and higher oligomers of ethylene glycol. Useful polyethylene glycols generally have a number-average molecular weight ranging from 50 to 600 g/mol, preferably from 55 to 400 g/mol and especially from 60 to 200 g/mol and specifically from 62 g/mol to 150 g/mol.

The amount in which polyether polyol b4) is used is preferably from 10 to 40 mol %, more preferably from 12 to 30 mol % and especially from 14 to 25 mol %, and is at least 200 mmol, preferably at least 400 mmol, more preferably at least 600 mmol, especially at least 800 mmol and specifically at least 1000 mmol of component b4) per kg of polyesterol B). Component b4) is preferably an alkoxylated triol or polyol, more preferably an alkoxylated triol and even more preferably a polyether prepared by the addition of ethylene oxide or propylene oxide, preferably ethylene oxide, onto glycerol or trimethylolpropane, preferably glycerol, as starter molecule. The use of ethylene oxide leads to rigid foams having improved fire behavior.

In a preferred embodiment of the present invention the starter molecule is alkoxylated to prepare component b4) by using a catalyst from the group consisting of potassium hydroxide (KOH) and aminic alkoxylation catalysts, in which case the use of aminic alkoxylation catalysts is preferred, since the polyetherols thus obtained can be used in the subsequent esterification without workup, while the polyether first has to be neutralized and separated off when KOH is used as alkoxylation catalyst. Preferred aminic alkoxylation catalysts are selected from the group consisting of dimethylethanolamine (DMEOA), imidazole and imidazole derivatives and also mixtures thereof, particular preference being given to imidazole.

In a specific embodiment of the invention, the polyether polyol b4) consists of the reaction product of glycerol with ethylene oxide and/or propylene oxide, preferably with ethylene oxide. The storage stability of component B) is particularly high as a result.

In a further specific embodiment of the invention, the polyether polyol b4) consists of the reaction product of trimethylolpropane with ethylene oxide and/or propylene oxide, preferably with ethylene oxide. This likewise results in a particularly high storage stability on the part of component B).

The OH number of polyether polyol b4) is preferably in the range from 150 to 1250 mg KOH/g, preferably from 300 to 950 mg KOH/g and more preferably from 500 to 800 mg KOH/g. In this range, particularly favorable mechanical properties and/or fire protection properties are achievable.

In a particularly preferred embodiment of the invention, the polyether polyol b4) consists of the reaction product of trimethylolpropane or glycerol, preferably glycerol, with ethylene oxide, the OH number of polyether polyol b4) is in the range from 500 to 800 mg KOH/g and preferably from 500 to 650 mg KOH/g, and imidazole is used as alkoxylation catalyst.

In an especially preferred embodiment of the invention, the polyether polyol b4) consists of the reaction product of trimethylolpropane or glycerol, preferably glycerol, with ethylene oxide, the OH number of polyether polyol b4) is in the range from 500 to 800 mg KOH/g and preferably from 500 to 650 mg KOH/g, imidazole is used as alkoxylation catalyst, the aliphatic or cycloaliphatic diol b3) is diethylene glycol, and component b2) is soybean oil, rapeseed oil or tallow, preferably tallow.

The number-weighted average functionality of polyester polyol B) is preferably not less than 2, more preferably greater than 2, even more preferably greater than 2.2 and especially greater than 2.3, which leads to a higher crosslink density on the part of the polyurethane produced therewith and hence to better mechanical properties on the part of the polyurethane foam.

Polyester polyols B) are obtainable by polycondensing components b1) to b4) in the absence of catalysts or preferably in the presence of esterification catalysts, advantageously in an atmosphere of inert gas such as nitrogen in the melt at temperatures of 150 to 280° C., preferably 180 to 260° C. optionally under reduced pressure to the desired acid number, which is advantageously less than 10 and preferably less than 2. In a preferred embodiment, the esterification mixture is polycondensed at the abovementioned temperatures to an acid number of 80 to 20, preferably 40 to 20, under atmospheric pressure and subsequently under a pressure of less than 500 mbar, preferably 40 to 400 mbar. Possible esterification catalysts include for example iron, cadmium, cobalt, lead, zinc, antimony, magnesium, titanium and tin catalysts in the form of metals, metal oxides or metal salts. However, the polycondensation can also be carried out in the liquid phase in the presence of diluent and/or entrainer agents, for example benzene, toluene, xylene or chlorobenzene, to remove the water of condensation by azeotropic distillation.

To prepare the polyester polyols B), the organic polycarboxylic acids and/or derivatives and polyhydric alcohols are advantageously polycondensed in a molar ratio of 1:1 to 2.2, preferably 1:1.05 to 2.1 and more preferably 1:1.1 to 2.0.

The resulting polyester polyols B) generally have a number-average molecular weight in the range from 300 to 3000, preferably in the range from 400 to 1000 and especially in the range from 450 to 800.

The proportion of polyester polyols B) according to the present invention is generally at least 10 wt %, preferably at least 20 wt %, more preferably at least 40 wt % and specifically at least 50 wt %, based on total components B) to H).

Rigid polyurethane foams are obtained according to the process of the present invention by using the specific polyester polyols B) described above alongside construction components known per se, which will now be discussed in detail.

### Component A

A polyisocyanate for the purposes of the present invention is an organic compound comprising two or more than two reactive isocyanate groups per molecule, i.e., isocyanate functionality is not less than 2. When the polyisocyanates used or a mixture of two or more polyisocyanates do not have a unitary functionality, the number-weighted average functionality of component A) will be not less than 2.

Useful polyisocyanates A) include the well-known aliphatic, cycloaliphatic, araliphatic and preferably aromatic polyfunctional isocyanates. Polyfunctional isocyanates of this type are known per se or are obtainable by methods known per se. Polyfunctional isocyanates can more particularly also be used as mixtures, in which case component A) comprises various polyfunctional isocyanates. The number of isocyanate groups per molecule in polyfunctional isocyanates useful as polyisocyanate is two (and so the polyfunctional isocyanates in question are referred to hereinbelow as diisocyanates) or more than two.

Particularly the following may be mentioned in detail: alkylene diisocyanates having 4 to 12 carbon atoms in the alkylene radical, such as 1,12-dodecane diioscyanate, 2-ethyltetramethylene 1,4-diisocyanate, 2-methylpentamethylene 1,5-diisocyanate, tetramethylene 1,4-diisocyanate, and preferably hexamethylene 1,6-diisocyanate; cycloaliphatic diisocyanates such as cyclohexane 1,3- and 1,4-diisocyanate and also any desired mixtures of these isomers, 1-isocyanato-3,3,5-trimethyl-5-isocyanatomethylcyclohexane (IPDI), 2,4- and 2,6-hexahydrotolylene diisocyanate and also the corresponding isomeric mixtures, 4,4′-, 2,2′- and 2,4′-dicyclohexylmethane diisocyanate and also the corresponding isomeric mixtures, and preferably aromatic polyisocyanates, such as 2,4- and 2,6-tolylene diisocyanate and the corresponding isomeric mixtures, 4,4′-, 2,4′- and 2,2′-diphenylmethane diisocyanate and the corresponding isomeric mixtures, mixtures of 4,4′- and 2,2′-diphenylmethane diisocyanates, polyphenylpolymethylene polyisocyanates, mixtures of 4,4′-, 2,4′- and 2,2′-diphenylmethane diisocyanates and polyphenylpolymethylene polyisocyanates (crude MDI) and mixtures of crude MDI and tolylene diisocyanates.

Of particular suitability are 2,2′-, 2,4′- and/or 4,4′-diphenylmethane diisocyanate (MDI), 1,5-naphthylene diisocyanate (NDI), 2,4- and/or 2,6-tolylene diisocyanate (TDI), 3,3′-dimethylbiphenyl diisocyanate, 1,2-diphenylethane diisocyanate and/or p-phenylene diisocyanate (PPDI), tri-, tetra-, penta-, hexa-, hepta- and/or octamethylene diisocyanate, 2-methylpentamethylene 1,5-diisocyanate, 2-ethylbutylene 1,4-diisocyanate, pentamethylene 1,5-diisocyanate, butylene 1,4-diisocyanate, 1-isocyanato-3,3,5-trinnethyl-5-isocyanatomethylcyclohexane (isophorone diisocyanate, IPDI), 1,4- and/or 1,3-bis(isocyanatomethyl)cyclohexane (HXDI), 1,4-cyclohexane diisocyanate, 1-methyl-2,4- and/or -2,6-cyclohexane diisocyanate and 4,4′-, 2,4′- and/or 2,2′-dicyclohexylmethane diisocyanate.

Frequent use is also made of modified polyisocyanates, i.e. products obtained by chemical conversion of organic polyisocyanates and having two or more than two reactive isocyanate groups per molecule. Polyisocyanates comprising ester, urea, biuret, allophanate, carbodiimide, isocyanurate, uretdione, carbamate and/or urethane groups may be mentioned in particular.

The following embodiments are particularly preferable as polyisocyanates of component A):
i) polyfunctional isocyanates based on tolylene diisocyanate (TDI), especially 2,4-TDI or 2,6-TDI or mixtures of 2,4- and 2,6-TDI;
ii) polyfunctional isocyanates based on diphenylmethane diisocyanat (MDI), especially 2,2′-MDI or 2,4′-MDI or 4,4′-MDI or oligomeric MDI, which is also known as polyphenylpolymethylene isocyanate, or mixtures of two or three aforementioned diphenylmethane diisocyanates, or crude MDI, which is obtained in the production of MDI, or mixtures of at least one oligomer of MDI and at least one aforementioned low molecular weight MDI derivative;
iii) mixtures of at least one aromatic isocyanate as per embodiment i) and at least one aromatic isocyanate as per embodiment ii).

Polymeric diphenylmethane diisocyanate is very particularly preferred as polyisocyanate. Polymeric diphenylmethane diisocyanate (called polymeric MDI hereinbelow) is a mixture of binuclear MDI and oligomeric condensation products and thus derivatives of diphenylmethane diisocyanate (MDI). Polyisocyanates may preferably also be constructed from mixtures of monomeric aromatic diisocyanates and polymeric MDI.

Polymeric MDI, in addition to binuclear MDI, comprises one or more polynuclear condensation products of MDI with a functionality of more than 2, especially 3 or 4 or 5. Polymeric MDI is known and often referred to as polyphenylpolymethylene isocyanate or else as oligomeric MDI. Polymeric MDI is typically constructed from a mixture of MDI-based isocyanates of differing functionality. Polymeric MDI is typically used in admixture with monomeric MDI.

The (average) functionality of a polyisocyanate comprising polymeric MDI can vary in the range from about 2.2 to about 5, especially from 2.3 to 4, especially from 2.4 to 3.5. Crude MDI, obtained as an intermediate in the production of MDI, is more particularly such a mixture of MDI-based polyfunctional isocyanates having different functionalities.

Polyfunctional isocyanates or mixtures of two or more polyfunctional isocyanates based on MDI are known and available for example from BASF Polyurethanes GmbH under the name of Lupranat®.

The functionality of component A) is preferably at least two, more preferably at least 2.2 and especially at least 2.4. The functionality of component A) is preferably from 2.2 to 4 and more preferably from 2.4 to 3.

The isocyanate group content of component A) is preferably from 5 to 10 mmol/g, more preferably from 6 to 9 mmol/g and especially from 7 to 8.5 mmol/g. A person skilled in the art is aware of a reciprocal relationship between the isocyanate group content in mmol/g and the so-called equivalence weight in g/equivalent. The isocyanate group content in mmol/g is obtained from the content in wt % according to ASTM D-5155-96 A.

In a particularly preferred embodiment, component A) consists of at least one polyfunctional isocyanate selected from diphenylmethane 4,4′-diisocyanate, diphenylmethane 2,4′-diisocyanate, diphenylmethane 2,2′-diisocyanate and oligomeric diphenylmethane diisocyanate. In this preferred embodiment, component A) more preferably comprises oligomeric diphenylmethane diisocyanate and has a functionality of at least 2.4.

The viscosity of component A) can vary within wide limits. The viscosity of component A) is preferably in the range from 100 to 3000 mPa·s and more preferably in the range from 200 to 2500 mPa·s.

### Component C

Useful polyester polyols C) differ from polyesterols B) and can be prepared, for example, from organic dicarboxylic acids having 2 to 12 carbon atoms, preferably aromatic ones, or mixtures of aromatic and aliphatic dicarboxylic acids, and polyhydric alcohols, preferably diols, having 2 to 12 carbon atoms and preferably 2 to 6 carbon atoms.

Possible dicarboxylic acids are, in particular: succinic acid, glutaric acid, adipic acid, suberic acid, azelaic acid, sebacic acid, decanedicarboxylic acid, maleic acid, fumaric acid, phthalic acid, isophthalic acid and terephthalic acid. It is likewise possible to use derivatives of these dicarboxylic acids, such as dimethyl terephthalate, for example. The dicarboxylic acids can be used either individually or in admixture with one another. It is also possible to use the corresponding dicarboxylic acid derivatives, e.g. dicarboxylic esters of alcohols having from 1 to 4 carbon atoms or dicarboxylic anhydrides, in place of the free dicarboxylic acids. As aromatic dicarboxylic acids, preference is given to using phthalic acid, phthalic anhydride, terephthalic acid and/or isophthalic acid as a mixture or alone. As aliphatic dicarboxylic acids, preference is given to using dicarboxylic acid mixtures of succinic, glutaric and adipic acid in weight ratios of, for example, 20-35:35-50:20-32 parts by weight and in particular adipic acid. Examples of dihydric and polyhydric alcohols, in particular diols, are: ethanediol, diethylene glycol, 1,2- or 1,3-propanediol, dipropylene glycol, 1,4-butanediol, 1,5-pentanediol, 1,6-hexanediol, 1,10-decanediol, glycerol, trimethylolpropane and pentaerythritol. Preference is given to using ethanediol, diethylene glycol, 1,4-butanediol, 1,5-pentanediol, 1,6-hexanediol or mixtures of at least two of the diols mentioned, in particular mixtures of 1,4-butanediol, 1,5-pentanediol and 1,6-hexanediol. It is also possible to use polyester polyols derived from lactones, e.g., E-caprolactone, or hydroxycarboxylic acids, e.g., ω-hydroxycaproic acid.

To prepare the further polyester polyols C), biobased starting materials and/or derivatives thereof are also suitable, for example, castor oil, polyhydroxy fatty acids, ricinoleic acid, hydroxyl-modified oils, grapeseed oil, black cumin oil, pumpkin kernel oil, borage seed oil, soybean oil, wheat germ oil, rapeseed oil, sunflower oil, peanut oil, apricot kernel oil, pistachio oil, almond oil, olive oil, macadamia nut oil, avocado oil, sea buckthorn oil, sesame oil, hemp oil, hazelnut oil, primula oil, wild rose oil, safflower oil, walnut oil, fatty acids, hydroxyl-modified fatty acids and fatty acid esters based on myristoleic acid, palmitoleic acid, oleic acid, vaccenic acid, petroselic acid, gadoleic acid, erucic acid, nervonic acid, linoleic acid, α- and γ-linolenic acid, stearidonic acid, arachidonic acid, timnodonic acid, clupanodonic acid and cervonic acid.

The mass ratio of polyesterols B) to the further polyester polyols C) is generally at least 0.1, preferably at least 0.5, more preferably at least 1.0 and especially at least 2.

One especially preferred embodiment does not utilize any further polyester polyols C).

### Component D

One or more polyether polyols D) can be used as component D. Polyetherols D) can be prepared by known methods, for example from one or more alkylene oxides having from 2 to 4 carbon atoms by anionic polymerization using alkali metal hydroxides, e.g., sodium or potassium hydroxide, or alkali metal alkoxides, e.g., sodium methoxide, sodium or potassium ethoxide or potassium isopropoxide, or aminic alkoxylation catalysts, such as dimethylethanolamine (DMEOA), imidazole and/or imidazole derivatives, with use of at least one starter molecule comprising from 2 to 8, preferably from 2 to 6, reactive hydrogen atoms in bonded form, or by cationic polymerization using Lewis acids, e.g., antimony pentachloride, boron fluoride etherate, or bleaching earth.

Suitable alkylene oxides are, for example, tetrahydrofuran, 1,3-propylene oxide, 1,2- or 2,3-butylene oxide, styrene oxide and preferably ethylene oxide and 1,2-propylene oxide. The alkylene oxides can be used individually, alternately in succession or as mixtures. Preferred alkylene oxides are propylene oxide and ethylene oxide, with particular preference being given to ethylene oxide.

Possible starter molecules are, for example: water, organic dicarboxylic acids, such as succinic acid, adipic acid, phthalic acid and terephthalic acid, aliphatic and aromatic, optionally N-monoalkyl-, N,N-dialkyl- and N,N′-dialkyl-substituted diamines having from 1 to 4 carbon atoms in the alkyl radical, e.g. optionally monoalkyl- and dialkyl-substituted ethylenediamine, diethylenetriamine, triethylenetetramine, 1,3-propylenediamine, 1,3- or 1,4-butylenediamine, 1,2-, 1,3-, 1,4-, 1,5- and 1,6-hexamethylenediamine, phenylenediamines, 2,3-, 2,4- and 2,6-tolylenediamine and 4,4′-, 2,4′- and 2,2′-diaminodiphenylmethane. Particular preference is given to the recited diprimary amines, for example ethylenediamine.

Further possible starter molecules are: alkanolamines such as ethanolamine, N-methylethanolamine and N-ethylethanolamine, dialkanolamines, such as diethanolamine, N-methyldiethanolamine and N-ethyldiethanolamine and trialkanolamines, e.g., triethanolamine, and ammonia.

Preference is given to using dihydric or polyhydric alcohols, e.g., ethanediol, 1,2- and 1,3-propanediol, diethylene glycol (DEG), dipropylene glycol, 1,4-butanediol, 1,6-hexanediol, glycerol, trimethylolpropane, pentaerythritol, sorbitol and sucrose.

Polyether polyols D), possibly polyoxypropylene polyols and polyoxyethylene polyols, more preferably polyoxyethylene polyols, have a functionality of preferably 2 to 6, more preferably 2 to 4, especially 2 to 3 and specifically 2 and number-average molecular weights of 150 to 3000 g/mol, preferably 200 to 2000 g/mol and especially 250 to 1000 g/mol.

One preferred embodiment of the invention utilizes an alkoxylated diol, preferably an ethoxylated diol, for example ethoxylated ethylene glycol, as polyether polyol D), preferably polyethylene glycol is concerned.

In a specific embodiment of the invention, the polyetherol component D) consists exclusively of polyethylene glycol, preferably with a number-average molecular weight of 250 to 1000 g/mol.

The proportion of polyether polyols D) is generally in the range from 0 to 11 wt %, preferably in the range from 2 to 9 wt % and more preferably in the range from 4 to 8 wt %, based on total components B) to H).

The mass ratio of total components B) and C) to component D) is generally greater than 1, preferably greater than 2, more preferably greater than 7, even more preferably greater than 10 and yet even more preferably greater than 12.

The mass ratio of total components B) and C) to component D) is further generally less than 80, preferably less than 40, more preferably less than 30, even more preferably less than 20, yet even more preferably less than 16 and specifically less than 13.

### Component E

As flame retardants E), it is generally possible to use the flame retardants known from the prior art. Suitable flame retardants are, for example, brominated esters, brominated ethers (Ixol) or brominated alcohols such as dibromoneopentyl alcohol, tribromoneopentyl alcohol and PHT-4-diol and also chlorinated phosphates such as tris(2-chloroethyl) phosphate, tris(2-chloropropyl) phosphate (TCPP), tris(1,3-dichloropropyl) phosphate, tricresyl phosphate, tris(2,3-dibromopropyl) phosphate, tetrakis(2-chloroethyl) ethylenediphosphate, dimethyl methanephosphonate, diethyl diethanolaminomethylphosphonate and also commercial halogen-comprising flame retardant polyols. By way of further phosphates or phosphonates it is possible to use diethyl ethanephosphonate (DEEP), triethyl phosphate (TEP), dimethyl propylphosphonate (DMPP) or diphenyl cresyl phosphate (DPK) as liquid flame retardants.

Apart from the abovementioned flame retardants, it is also possible to use inorganic or organic flame retardants such as red phosphorus, preparations comprising red phosphorus, aluminum oxide hydrate, antimony trioxide, arsenic oxide, ammonium polyphosphate and calcium sulfate, expandable graphite or cyanuric acid derivatives such as melamine, or mixtures of at least two flame retardants, e.g. ammonium polyphosphates and melamine and optionally maize starch or ammonium polyphosphate, melamine, expandable graphite and optionally aromatic polyesters for making the rigid polyurethane foams flame resistant.

Preferable flame retardants have no isocyanate-reactive groups. The flame retardants are preferably liquid at room temperature. Particular preference is given to TCPP, DEEP, TEP, DMPP and DPK.

The proportion of flame retardant E) is generally in the range from 2 to 50 wt %, preferably in the range from 5 to 30 wt % and more preferably in the range from 8 to 25 wt %, based on components B) to H).

### Component F

Blowing agents F) which are used for producing the rigid polyurethane foams include preferably water, formic acid and mixtures thereof. These react with isocyanate groups to form carbon dioxide and in the case of formic acid carbon dioxide and carbon monoxide. Since these blowing agents release the gas through a chemical reaction with the isocyanate groups, they are termed chemical blowing agents. In addition, physical blowing agents such as low-boiling hydrocarbons can be used. Suitable in particular are liquids which are inert towards the polyisocyanates A) and have boiling points below 100° C., preferably below 50° C., at atmospheric pressure, so that they vaporize under the conditions of the exothermic polyaddition reaction. Examples of such liquids which can preferably be used are alkanes such as heptane, hexane, n-pentane and isopentane, preferably industrial mixtures of n-pentane and isopentane, n-butane and isobutane and propane, cycloalkanes such as cyclopentane and/or cyclohexane, ethers such as furan, dimethyl ether and diethyl ether, ketones such as acetone and methyl ethyl ketone, alkyl carboxylates such as methyl formate, dimethyl oxalate and ethyl acetate and halogenated hydrocarbons such as methylene chloride, dichloromonofluoromethane, difluoromethane, trifluoromethane, difluoroethane, tetrafluoroethane, chlorodifluoroethanes, 1,1-dichloro-2,2,2-trifluoroethane, 2,2-dichloro-2-fluoroethane and heptafluoropropane. Mixtures of these low-boiling liquids with one another and/or with other substituted or unsubstituted hydrocarbons can also be used. Organic carboxylic acids such as formic acid, acetic acid, oxalic acid, ricinoleic acid and carboxyl-containing compounds are also suitable.

It is preferable not to use any halogenated hydrocarbons as blowing agents. It is preferable to use water, formic acid-water mixtures or formic acid as chemical blowing agents and formic acid-water mixtures or formic acid are particularly preferred chemical blowing agents. Pentane isomers or mixtures of pentane isomers are preferably used as physical blowing agents.

The chemical blowing agents can be used alone, i.e., without addition of physical blowing agents, or together with physical blowing agents. Preferably, the chemical blowing agents are used together with physical blowing agents, in which case formic acid-water mixtures or pure formic acid together with pentane isomers or mixtures of pentane isomers are preferred.

The blowing agents are either wholly or partly dissolved in the polyol component (i.e. B+C+D+E+F+G+H) or are introduced via a static mixer immediately before foaming of the polyol component. It is usual for water, formic acid-water mixtures or formic acid to be fully or partially dissolved in the polyol component and the physical blowing agent (for example pentane) and any remainder of the chemical blowing agent to be introduced “on-line”.

The polyol component is admixed in situ with pentane, possibly some of the chemical blowing agent and also with all or some of the catalyst. Auxiliary and admixture agents as well as flame retardants are already comprised in the polyol blend.

The amount of blowing agent or blowing agent mixture used is in the range from 1 to 45 wt %, preferably in the range from 1 to 30 wt % and more preferably in the range from 1.5 to 20 wt %, all based on total components B) to H).

When water, formic acid or a formic acid-water mixture is used as blowing agent, it is preferably added to the polyol component (B+C+D+E+F+G+H) in an amount of 0.2 to 10 wt %, based on component B). The addition of water, formic acid or formic acid-water mixture can take place in combination with the use of other blowing agents described. Preference is given to using formic acid or a formic acid-water mixture in combination with pentane.

### Component G

Catalysts G) used for preparing the rigid polyurethane foams are particularly compounds which substantially speed the reaction of the components B) to H) compounds comprising reactive hydrogen atoms, especially hydroxyl groups, with the polyisocyanates A).

It is advantageous to use basic polyurethane catalysts, for example tertiary amines such as triethylamine, tributylamine, dimethylbenzylamine, dicyclohexylmethylamine, dimethylcyclohexylamine, N,N,N′,N′-tetramethyldiaminodiethyl ether, bis(dimethylaminopropyl)urea, N-methylmorpholine or N-ethylmorpholine, N-cyclohexylmorpholine, N,N,N′,N′-tetramethylethylenediamine, N,N,N,N-tetramethylbutanediamine, N,N,N,N-tetramethylhexane-1,6-diamine, pentamethyldiethylenetriamine, bis(2-dimethylaminoethyl) ether, dimethyl-piperazine, N-dimethylaminoethylpiperidine, 1,2-dimethylimidazole, 1-azabicyclo[2.2.0]octane, 1,4-diazabicyclo[2.2.2]octane (Dabco) and alkanolamine compounds, such as triethanolamine, triisopropanolamine, N-methyldiethanolamine and N-ethyldiethanolamine, dimethylaminoethanol, 2-(N,N-dimethylaminoethoxy)ethanol, N,N′,N″-tris(dialkylaminoalkyl)hexahydrotriazines, e.g. N,N′,N″-tris(dimethylaminopropyl)-s-hexahydrotriazine, and triethylenediamine. However, metal salts such as iron(II) chloride, zinc chloride, lead octoate and preferably tin salts such as tin dioctoate, tin diethylhexoate and dibutyltin dilaurate and also, in particular, mixtures of tertiary amines and organic tin salts are also suitable.

Further possible catalysts are: amidines such as 2,3-dimethyl-3,4,5,6-tetrahydropyrimidine, tetraalkylammonium hydroxides such as tetramethylammonium hydroxide, alkali metal hydroxides such as sodium hydroxide and alkali metal alkoxides such as sodium methoxide and potassium isopropoxide, alkali metal carboxylates and also alkali metal salts of long-chain fatty acids having from 10 to 20 carbon atoms and optionally lateral OH groups. Preference is given to using from 0.001 to 10 parts by weight of catalyst or catalyst combination, based (i.e., reckoned) on 100 parts by weight of component B). It is also possible to allow the reactions to proceed without catalysis. In this case, the catalytic activity of amine-initiated polyols is exploited.

When, during foaming, a relatively large polyisocyanate excess is used, further suitable catalysts for the trimerization reaction of the excess NCO groups with one another are: catalysts which form isocyanurate groups, for example ammonium ion salts or alkali metal salts, specifically ammonium or alkali metal carboxylates, either alone or in combination with tertiary amines. Isocyanurate formation leads to flame-resistant PIR foams which are preferably used in industrial rigid foam, for example in building and construction as insulation boards or sandwich elements.

Further information regarding the abovementioned and further starting materials may be found in the technical literature, for example Kunststoffhandbuch, Volume VII, Polyurethane, Carl Hanser Verlag Munich, Vienna, 1st, 2nd and 3rd Editions 1966, 1983 and 1993.

### Component H

Further auxiliaries and/or admixture agents H) can optionally be added to the reaction mixture for producing the rigid polyurethane foams. Mention may be made of, for example, surface-active substances, foam stabilizers, cell regulators, fillers, dyes, pigments, hydrolysis inhibitors, fungistatic and bacteriostatic substances.

Possible surface-active substances are, for example, compounds which serve to aid homogenization of the starting materials and may also be suitable for regulating the cell structure of the polymers. Mention may be made of, for example, emulsifiers such as the sodium salts of castor oil sulfates or of fatty acids and also salts of fatty acids with amines, e.g. diethylamine oleate, diethanolamine stearate, diethanolamine ricinoleate, salts of sulfonic acids, e.g. alkali metal or ammonium salts of dodecylbenzenedisulfonic or dinaphthylmethanedisulfonic acid and ricinoleic acid; foam stabilizers such as siloxane-oxyalkylene copolymers and other organopolysiloxanes, ethoxylated alkylphenols, ethoxylated fatty alcohols, paraffin oils, castor oil esters or ricinoleic esters, Turkey red oil and peanut oil, and cell regulators such as paraffins, fatty alcohols and dimethylpolysiloxanes. The above-described oligomeric acrylates having polyoxyalkylene and fluoroalkane radicals as side groups are also suitable for improving the emulsifying action, the cell structure and/or for stabilizing the foam. The surface-active substances are usually employed in amounts of from 0.01 to 10 parts by weight, based (i.e., reckoned) on 100 parts by weight of the component B).

For the purposes of the present invention, fillers, in particular reinforcing fillers, are the customary organic and inorganic fillers, reinforcing materials, weighting agents, agents for improving the abrasion behavior in paints, coating compositions, etc., which are known per se. Specific examples are: inorganic fillers such as siliceous minerals, for example sheet silicates such as antigorite, serpentine, hornblendes, amphiboles, chrisotile and talc, metal oxides such as kaolin, aluminum oxides, titanium oxides and iron oxides, metal salts, such as chalk, barite and inorganic pigments such as cadmium sulfide and zinc sulfide and also glass, etc. Preference is given to using kaolin (china clay), aluminum silicate and coprecipitates of barium sulfate and aluminum silicate and also natural and synthetic fibrous minerals such as wollastonite, metal fibers and in particular glass fibers of various length, which may be coated with a size. Possible organic fillers are, for example: carbon, melamine, rosin, cyclopentadienyl resins and graft polymers and also cellulose fibers, polyamide, polyacrylonitrile, polyurethane, polyester fibers based on aromatic and/or aliphatic dicarboxylic esters and in particular carbon fibers.

The inorganic and organic fillers can be used individually or as mixtures and are advantageously added to the reaction mixture in amounts of from 0.5 to 50 wt %, preferably from 1 to 40 wt %, based on the weight of the components A) to H), although the content of mats, nonwovens and woven fabrics of natural and synthetic fibers can reach values of up to 80 wt %, based on the weight of components A) to H).

Further information regarding the abovementioned other customary auxiliary and admixture agents may be found in the technical literature, for example the monograph by J. H. Saunders and K. C. Frisch “High Polymers” Volume XVI, Polyurethanes, Parts 1 and 2, Interscience Publishers 1962 and 1964, or Kunststoff-Handbuch, Polyurethane, Volume VII, Hanser-Verlag, Munich, Vienna, 1st and 2nd Editions, 1966 and 1983.

The present invention further provides a polyol component comprising:

from 10 to 90 wt % of polyesterols B),

from 0 to 60 wt % of further polyester polyols C),

from 0 to 11 wt % of polyether polyols D),

from 2 to 50 wt % of flame retardants E),

from1 to 45 wt % of blowing agents F),

from 0.001 to 10 wt % of catalysts G), and

from 0.5 to 20 wt % of further auxiliary and admixture agents H),

each as defined above and each based on the total weight of components B) to H), wherein the wt % sum to 100 wt %, and wherein the mass ratio of total components B) and C) to component D) is at least 1.

It is particularly preferable for the polyol component to comprise

from 50 to 90 wt % of polyesterols B),

from 0 to 20 wt % of further polyester polyols C),

from 2 to 9 wt % of polyether polyols D),

from 5 to 30 wt % of flame retardants E),

from 1 to 30 wt % of blowing agents F),

from 0.5 to 10 wt % of catalysts G), and

from 0.5 to 20 wt % of further auxiliary and admixture agents H),

each as defined above and each based on the total weight of components B) to H), wherein the wt % sum to 100 wt %, and wherein the mass ratio of total components B) and C) to component D) is at least 2.

The mass ratio of total components B) and optionally C) to component D) in the polyol components of the present invention is further generally less than 80, preferably less than 40, more preferably less than 30, even more preferably less than 20, yet even more preferably less than 16 and most preferably less than 13.

To produce the rigid polyurethane foams of the invention, the optionally modified organic polyisocyanates A), the specific polyester polyols B) of the invention, optionally the further polyester polyols C), the polyetherols D) and the further components E) to H) are mixed in such amounts that the equivalence ratio of NCO groups of the polyisocyanates A) to the sum of the reactive hydrogen atoms of the components B) and optionally C) and D) to H) is 1-6:1, preferably 1.6-5:1 and in particular 2.5-3.5:1.

The examples which follow illustrate the invention.

### EXAMPLES

### Polyesterol 1 (Not In Accordance With the Present Invention)

Esterification product of terephthalic acid (30.5 mol %), oleic acid (9.2 mol %), diethylene glycol (36.6 mol %) and a polyether polyol (23.7 mol %) based on trimethylolpropane and ethylene oxide with an OH functionality of 3 and a hydroxyl number of 610 mg KOH/g, prepared in the presence of imidazole as alkoxylation catalyst. The polyether was used in the subsequent esterification without workup. Polyesterol 1 had a hydroxyl functionality of 2.49 and a hydroxyl number of 245 mg KOH/.

### Polyesterol 2 (In Accordance With the Present Invention)

Esterification product of terephthalic acid (35.4 mol %), soybean oil (2.1 mol %), diethylene glycol (44.3 mol %) and a polyether polyol (18.2 mol %) based on trimethylolpropane and ethylene oxide with an OH functionality of 3 and a hydroxyl number of 610 mg KOH/g, prepared in the presence of imidazole as alkoxylation catalyst. The polyether polyol was used in the subsequent esterification without workup. Polyesterol 2 had a hydroxyl functionality of 2.48 and a hydroxyl number of 251 mg KOH/.

### Polyesterol 3 (In Accordance With the Present Invention)

Esterification product of terephthalic acid (36.0 mol %), soybean oil (1.4 mol %), diethylene glycol (46.9 mol %) and a polyether polyol (15.7 mol %) based on trimethylolpropane and ethylene oxide with an OH functionality of 3 and a hydroxyl number of 610 mg KOH/g, prepared in the presence of imidazole as alkoxylation catalyst. The polyether polyol was used in the subsequent esterification without workup. Polyesterol 3 had a hydroxyl functionality of 2.46 and a hydroxyl number of 253 mg KOH/.

### Polyesterol 4 (Not In Accordance With the Present Invention)

Esterification product of terephthalic acid (37.0 mol %), soybean oil (0.7 mol %), diethylene glycol (48.2 mol %) and a polyether polyol (14.1 mol %) based on trimethylolpropane and ethylene oxide with an OH functionality of 3 and a hydroxyl number of 610 mg KOH/g, prepared in the presence of imidazole as alkoxylation catalyst. The polyether polyol was used in the subsequent esterification without workup. Polyesterol 4 had a hydroxyl functionality of 2.49 and a hydroxyl number of 250 mg KOH/.

### Determination of Processability

Processability was determined by observing the foam formation process. Large bubbles of blowing agent which burst at the surface of the foam and thus tear open the surface of the foam were referred to as “blowouts” and the system was classed as not satisfactorily processable. If this unsatisfactory behavior was not observed, processability was classed as satisfactory.

### Smoke Gas Production

Smoke gas production was measured in a cone calorimeter using a helium-neon laser and a photodiode and determined in accordance with ISO 5660-2 as total smoke production [m²/m²] and average specific extinction area (ASEA) [m²/kg].

### Production of Rigid Polyurethane Foams (Variant 1)

The isocyanates and the isocyanate-reactive components were foamed up at a constant polyol component/isocyanate mixing ratio of 100:160 together with the blowing agents, catalysts and all further admixture agents.

### Polyol Component

40.0 parts by weight of polyesterol as per inventive or comparative examples;
27.0 parts by weight of polyether polyol with OH number about 490 mg KOH/g, prepared by polyaddition of propylene oxide onto a sucrose-glycerol mixture as starter molecule (66.4 wt % PO, 20.3 wt % sucrose, 13.3 wt % glycerol);
5.5 parts by weight of polyetherol consisting of the ether of ethylene glycol and ethylene oxide with hydroxyl functionality 2 and hydroxyl number 200 mg KOH/g;
25 parts by weight of trischlorisopropyl phosphate (TCPP) as flame retardant;
2.5 parts by weight of Niax Silicone L 6635 stabilizer; additives to polyol component:
5.5 parts by weight of Pentane S 80:20 (80 wt % n-pentane and 20 wt % isopentane);
about 2.6 parts by weight of water;
1.5 parts by weight of potassium acetate solution (47 wt % in ethylene glycol);
about 1.1 parts by weight of dimethylcyclohexylamine

### Isocyanate Component

160 parts by weight of Lupranat® M50 (polymeric methylenediphenyl diisocyanate (PMDI) with viscosity about 500 mPa·s at 25° C.).

Sandwich elements 50 mm thick were produced by the double belt process. Foam density was adjusted to 38 +/−1 g/L by varying the water content while keeping the pentane content constant at 5.5 parts. Fiber time was controlled to 25 +/−1 s by varying the proportion of dimethylcyclohexylamine.

### Production of Rigid Polyurethane Foams (Variant 2)

The isocyanates and the isocyanate-reactive components were foamed up at a constant polyol/isocyanate mixing ratio of 100:180 together with the blowing agents, catalysts and all further admixture agents.

### Polyol Component

40.0 parts by weight of polyesterol as per inventive or comparative examples;
27.0 parts by weight of polyether polyol with OH number about 490 mg KOH/g, prepared by polyaddition of propylene oxide onto a sucrose-glycerol mixture as starter molecule (composition like variant 1);
5.5 parts by weight of polyetherol consisting of the ether of ethylene glycol and ethylene oxide with hydroxyl functionality 2 and hydroxyl number 200 mg KOH/g;
25 parts by weight of trischlorisopropyl phosphate (TCPP) as flame retardant;
2.5 parts by weight of Niax Silicone L 6635 stabilizer; additives to polyol component:
5.5 parts by weight of Pentane S 80:20 (80 wt % n-pentane and 20 wt % isopentane);
about 2.8 parts by weight of water;
1.5 parts by weight of potassium acetate solution (47 wt % in ethylene glycol);
about 1.3 parts by weight of dimethylcyclohexylamine.

### Isocyanate Component

180 parts by weight of Lupranat® M50 (polymeric methylenediphenyl diisocyanate (PMDI) with viscosity about 500 mPa·s at 25° C.).

Sandwich elements 50 mm thick were produced by the double belt process. Foam density was adjusted to 38 +/−1 g/L by varying the water content while keeping the pentane content constant at 5.5 parts. Fiber time was controlled to 25 +/−1 s by varying the proportion of dimethylcyclohexylamine.

### Production of Rigid Polyurethane Foams (Variant 3)

The isocyanates and the isocyanate-reactive components were foamed up at a constant polyol/isocyanate mixing ratio of 100:200 together with the blowing agents, catalysts and all further admixture agents.

### Polyol Component

40.0 parts by weight of polyesterol as per inventive or comparative examples;
27.0 parts by weight of polyether polyol with OH number about 490 mg KOH/g, prepared by polyaddition of propylene oxide onto a sucrose-glycerol mixture as starter molecule (composition like variant 1);
5.5 parts by weight of polyetherol consisting of the ether of ethylene glycol and ethylene oxide with hydroxyl functionality 2 and hydroxyl number 200 mg KOH/g;
25 parts by weight of trischlorisopropyl phosphate (TCPP) as flame retardant;
2.5 parts by weight of Niax Silicone L 6635 stabilizer; additives to polyol component:
5.5 parts by weight of Pentane S 80:20 (80 wt % n-pentane and 20 wt % isopentane;
about 3.1 parts by weight of water;
1.5 parts by weight of potassium acetate solution (47 wt % in ethylene glycol);
about 1.5 parts by weight of dimethylcyclohexylamine.

### Isocyanate Component

200 parts by weight of Lupranat® M50 (polymeric methylenediphenyl diisocyanate (PMDI) with viscosity about 500 mPa·s at 25° C.).

Sandwich elements 50 mm thick were produced by the double belt process. Foam density was adjusted to 38 +/−1 g/L by varying the water content while keeping the pentane content constant at 5.5 parts. Fiber time was controlled to 25 +/−1 s by varying the proportion of dimethylcyclohexylamine.

The results are summarized in Table 1.

TABLE 1
Results of attempts to produce 50 mm thick sandwich elements by
double belt process

| | | | |
|---|---|---|---|
| Version | 1 | 2 | 3 |
| mixing ratio | 160 | 180 | 200 |

polyesterol 1

| | | | |
|---|---|---|---|
| visual assessment | good | good | good |
| processing | satisfactory | satisfactory | satisfactory |

polyesterol 2

| | | | |
|---|---|---|---|
| visual assessment | good | good | good |
| processing | satisfactory | satisfactory | satisfactory |

polyesterol 3

| | | | |
|---|---|---|---|
| visual assessment | good | good | surface defects |
| processing | satisfactory | satisfactory | blowouts |

polyesterol 4

| | | | |
|---|---|---|---|
| visual assessment | surface defects | surface defects | surface defects |
| processing | blowouts | blowouts | blowouts |

Table 1 shows that the processing properties of inventive rigid polyurethane foams improve as the proportion of fatty acid triglyceride increases in the polyesterol used. The rigid foams produced from polyesterols 1 and 2 were obtained in all variants, i.e., with all mixing ratios (160/180/200), in a satisfactory manner with good surface appearance. The rigid foam produced from polyester 3 was only obtained with surface defects and in an unsatisfactory manner in the case of variant 3 (mixing ratio 200). The rigid foam produced from polyester 4 could not be satisfactorily obtained in any variant or any mixing ratio. The elements from all three variants had distinct surface defects.

TABLE 2
Results of smoke production from cone calorimeter tests to
ISO 5660 Parts 1 and 2 with foam samples from 50 mm thick
sandwich elements produced by double belt process

| | |
|---|---|
| Variant | 1 |
| mixing ratio | 160 |

polyesterol 1

| | |
|---|---|
| total smoke production [m²/m²] | 990 |
| ASEA [m²/kg] | 523 |

polyesterol 2

| | |
|---|---|
| total smoke production [m²/m²] | 929 |
| ASEA [m²/kg] | 509 |

polyesterol 3

| | |
|---|---|
| total smoke production [m²/m²] | 832 |
| ASEA [m²/kg] | 457 |

polyesterol 4

| | |
|---|---|
| total smoke production [m²/m²] | 650 |
| ASEA [m²/kg] | 363 |

Table 2 shows that smoke gas production decreases with decreasing fatty acid triglyceride content of polyesterol used.

We claim:

1. A polyesterol obtainable by reaction of
b1) from 10 to 70 mol % of at least one compound selected from the group consisting of terephthalic acid, dimethyl terephthalate, polyethylene terephthalate, phthalic anhydride, phthalic acid and isophthalic acid,
b2) from 0.8 to 4.5 mol % of a fatty acid triglyceride,
b3) from 10 to 70 mol % of a diol selected from the group consisting of ethylene glycol, diethylene glycol and polyethylene glycols,
b4) from 5 to 50 mol % of a polyether polyol having a functionality above 2, wherein at least 200 mmol of component b4) are used per kg of the polyesterol,
wherein the sum total of components b1) to b4) is 100 mol %.

2. The polyesterol according to claim 1 wherein said polyether polyol b4) is a polyether polyol having a functionality above 2 and is obtained by alkoxylating a polyol having a functionality above 2.

3. The polyesterol according to claim 1 wherein said polyether polyol b4) is obtained by alkoxylating a triol selected from the group consisting of trimethylolpropane, glycerol and mixtures thereof.

4. The polyesterol according to claim 1 wherein said polyether polyol b4) is obtained by alkoxylation with ethylene oxide.

5. The polyesterol according to claim 4 wherein said polyether polyol b4) is obtained by alkoxylation with ethylene oxide in the presence of an aminic alkoxylation catalyst.

6. The polyesterol according to claim 1 wherein said component b1) is selected from the group consisting of terephthalic acid, dimethyl terephthalate, polyethylene terephthalate, phthalic anhydride and phthalic acid.

7. The polyesterol according to claim 1 wherein said fatty acid triglyceride b2) is selected from the group consisting of soybean oil, rapeseed oil, tallow and mixtures thereof.

8. The polyesterol according to claim 1 wherein said diol b3) is diethylene glycol.

9. The polyesterol according to claim 1 wherein at least 400 mmol of polyether polyol b4) are used per kg of polyesterol.

10. The polyesterol according to claim 1 having an aaverage functionality of not less than 2.

11. A process for producing rigid polyurethane foams or rigid polyisocyanurate foams comprising the reaction of
A) at least one polyisocyanate,
B) at least one polyesterol according to claim 1
C) optionally one or more further polyester polyols other than those of component B),
D) optionally one or more polyether polyols,
E) optionally one or more flame retardants,
F) one or more blowing agents,
G) one or more catalysts, and
H) optionally further auxiliaries or admixture agents.

12. The process according to claim 11 wherein the mass ratio of total components B) and C) to component D) is at least 1.

13. The process according to claim 11 wherein the mass ratio of total components B) and C) to component D) is below 80.

14. The process according to claim 11 wherein the mass ratio of polyesterols B) to the further polyester polyols C) is at least 0.1.

15. The process according to claim 11 wherein no further polyester polyols C) are used.

16. The process according to claim 12 wherein only polyethylene glycol is used as component D).

17. A rigid polyurethane foam obtainable by the process according to claim 12.

18. A polyol component comprising
B) from 10 to 90 wt % of polyesterols according to claim 1,
C) from 0 to 60 wt % of further polyester polyols C) other than those of component B),
) from 0 to 11 wt % of polyether polyols,
E) from 2 to 50 wt % of flame retardants,
F) from 1 to 45 wt % of blowing agents,
G) from 0.001 to 10 wt % of catalysts, and
H) from 0.5 to 20 wt % of further auxiliary and admixture agents,
wherein the sum total of components B) to H) is 100 wt % and wherein the mass ratio of total components B) and C) to component D) is at least 1.
